(19) United States
(12) Patent Application Publication
(10) Pub. No.: US 2026/0026928 A1
(43) Pub. Date: Jan. 29, 2026

(54) METHOD OF MANUFACTURING AN INTRAOCULAR LENS

(71) Applicant: JelliSee Ophthalmics Inc., McLean, VA (US)

(72) Inventors: Forrest J. Ellis, McLean, VA (US); Nestor Farmiga, Rochester, NY (US)

(21) Appl. No.: 19/280,469

(22) Filed: Jul. 25, 2025

Related U.S. Application Data
(60) Provisional application No. 63/675,528, filed on Jul. 25, 2024.

(51) Int. Cl. A61F 2/16; B29C 65/00; B29C 65/48; B29K 683/00

(52) U.S. Cl. CPC A61F 2/1648; A61F 2/1635; B29C 65/483; B29C 66/24221; B29C 66/71; B29C 66/73366; B29K 2683/00

(57) ABSTRACT

A method of manufacturing an intraocular lens (IOL). Method includes mounting the anterior part of the IOL on a first nest where the anterior part defines a receptacle and has a haptic extending from an anterior face. A pre-determined desired amount of fluid is introduced in the receptacle. A bonding agent can be introduced into a trough of posterior part of the IOL and the posterior part can be mounted on a second nest. Force can be applied to draw the first nest towards the second nest (or vice versa) or the first and second nests can be placed in a vacuum sealed chamber and the first and second nest can be mechanically drawn together. Alternatively, the first and second nests can be placed in an inert gas chamber such that the posterior part and the anterior part are mechanically join together to form a fluid filled integral IOL.

### CROSS-REFERENCE TO RELATED APPLICATIONS

The present application claims priority to U.S. Provisional Application No. 63/675,528 filed on Jul. 25, 2024, which is incorporated by reference in its entirety.

### TECHNICAL FIELD

The present technology relates to a method of manufacturing an intraocular lens including an accommodating intraocular lens.

### BACKGROUND

The lens of the eye constitutes cells arranged in a lamellar manner and is divided into a central nucleus and a peripheral cortex. The lens is enclosed by a cellular basement membrane (the lens capsule). Along with the cornea, the lens focuses light (refraction) onto the retina of the eye. Refractive power is measured in diopters. The lens contributes approximately a third of the refractive power of the eye and it is responsible for fine-tuning the eye's focus so that objects over a wide range of distances can be seen clearly. The process whereby the lens changes the focus of the eye is called accommodation. Accommodation is measured in diopters of accommodation change. Accommodation occurs with contraction of the ciliary muscle, which reduces force on the lens suspensory zonular fibers (zonules). The lens zonules extend from the ciliary muscle to the near equatorial regions of the lens and suspend the lens behind the iris. Reduction of force on the zonules allows the lens of the eye to assume its natural, less stressed, more spherical shape; thereby increasing its dioptric power, and this brings objects at near into focus. The change in focus from distance focus to near focus is generally smooth and controlled by a complex neural feedback mechanism. The relative contribution to accommodation of the lens zonules, the lens capsule, the lens nucleus and the surrounding lens cortex is the subject of ongoing research.

With age, there is a gradual reduction in the ability of the lens to alter the focus of the eye (presbyopia). This manifests in a gradual loss of capacity to focus on near objects, typically requiring optical assistance (e.g. reading glasses, bifocals) beginning around the fifth to sixth decades of life. There are many theories explaining accommodation and the loss of accommodation. It is generally accepted that with aging the lens substance itself becomes more resistant to deformational change (e.g. less pliable, more rigid, or firmer). A comprehensive and definitive understanding of accommodation and the cause(s) of presbyopia is still subject to scientific inquiry. However, much is already known regarding structural and functional changes of the lens and the other structures of the eye that contribute to accommodation and to the gradual loss of accommodation.

The ciliary muscle has been shown to function throughout life without significant loss of function with aging. The lens zonules include anterior fibers which attach to the lens anterior to the lens equator, equatorial fibers which attach at the lens equator, and posterior fibers which attach posterior to the lens equator. It has been demonstrated that the anterior fibers primarily alter the shape of the anterior lens surface and the posterior fibers primarily control the shape of the posterior lens surface. The sparse and less robust equatorial fibers play a lesser role in changing of the shape of the surfaces of the lens. Some conditions and genetic disorders may contribute to lens zonule loss or breakage, but in general the lens zonules remain functional throughout life. Therefore, changes in the lens zonules are not considered a significant factor in the loss of accommodation.

The lens nucleus and cortex are unique in that the cellular makeup and structure of the lens allows for reshaping of individual cells with intracellular flow of cytosol (the fluid within the cell). As a result, the individual cell shape can change and cumulatively the entire lens contents, contained within the lens capsule, can be considered a viscoelastic or “semi-flowable fluid”. However, in youth, the lens nucleus is less resistant to deformational change (e.g. less firm or more pliable), than compared to the lens cortex. This is termed the lens elastic gradient. Studies have shown that with aging the lens nucleus becomes more resistant to deformational change (e.g. less pliable, or firmer). Likewise, studies show that the lens cortex also becomes more resistant to deformational change (e.g. less pliable or firmer with aging). However, the lens nucleus becomes firmer at a faster rate than the lens cortex. The crossover, where the nucleus becomes firmer, less pliable, or more resistant to deformational change than the lens cortex occurs around age 40-50 years of age. This relative change in the difference in firmness of the nucleus relative to that of the cortex correlates with the loss of accommodation and the onset of presbyopia.

The human crystalline lens can be affected by one or more disorders or conditions that reduces its function and/or reduces the clarity of the lens. A common condition that occurs with aging is the gradual opacification and reduced transparency of the lens of the eye. This condition is termed a cataract. Surgical removal of a cataractous lens and placement of an artificial replacement lens (such as an intraocular lens (“IOL”)) within the eye is a common surgical procedure. The development of a suitable IOL that can provide the optical quality and accommodation provided by the youthful biological lens has not been developed.

There are generally two classes of IOLs that have been developed that attempt to overcome the lack of accommodation of an IOL used to replace the natural lens when cataract surgery is performed: pseudo-accommodating lenses and accommodating lenses. A pseudo-accommodating lens can be a multiple focal point lens that uses a ring for distance focus and one or more center optics for intermediate and near focus. Other designs use diffraction optics to obtain a range of focus or use optics to achieve an extended depth of focus (EDOF). Multi-focus optics, diffraction optics, and EDOF optic IOLs can result in disruptive optical aberrations such as glare, halos, reduced contrast sensitivity, etc. Centration of these lenses within the capsular bag is important to their best visual function. These lenses use non-deforming optical elements and do not achieve the visual quality of a natural, youthful lens of the human eye. The accommodating class of IOLs includes a silicone elastomeric hinged lens that allows forward movement of the optic when the eye focuses at near. These lenses are typically placed in the lens capsular bag (the remaining thin layer of basement membrane that is the outermost layer of the natural lens and is typically left in place when the contents of the lens are removed during cataract surgery). Due to progressive fibrosis and stiffening of the lens capsule following cataract removal, the effective accommodation with these lenses is known to diminish over time. Overall, these lenses may be adequate for distance and intermediate vision, but only provide accommodation of about two diopters at most and this value has been shown to diminish over time.

Shaped haptics, levers, or other mechanical elements have been described to translate the compressive force exerted by the elasticity of the lens capsule and/or the radial compressive force exerted by the ciliary muscles to effect a desired axial displacement of the IOL optic. Additional examples may also provide flexible hinge regions of the haptic to facilitate axial displacement of an IOL along the optical axis. Several examples include annular ring elements in contact with the lens capsule and that use the compression of applied force by the capsule to effect axial displacement of the IOL optic. However, these IOLs are configured to be generally of fixed optical power and in line with the optical axis of the eye. As such, the axial displacement of the optical elements of these IOLs that is possible limits the dioptric power change attained. Some single or multiple optic lenses have incorporated a shape changing and axial displacement changing combination of lenses, such as a shape changing optic coupled to zonular contact haptics whereby compression of the lens capsule during accommodation results in both anterior displacement of the flexible optic along the optical axis, as well as compression of the sides of the optic at the equator. Other described IOLs rely on a posterior flexible region separated from a flexible anterior lens by an articulating member about the circumference.

It is known that the lens capsule, following cataract surgery, becomes less pliable and more fibrotic. IOLs that rely on retained capsular elasticity/pliability are unlikely to retain accommodating/dis-accommodating ability.

Surface shape changing lenses are more likely to result in greater degrees of dioptric power change. These lenses include lenses with fluid filled chambers that rely on compression by the lens capsule along the optical axis to force fluid from a peripheral chamber into a central lens and thereby change the shape of the central lens and therefore the optical power of the lens. Other lenses use the compressive force by the lens capsule to provide a radial inward compressive force about the equatorial periphery of a flexible lens to shape change the lens. These are generally two-part systems with a circumferential haptic design with a central fixed posterior lens that fits within the capsule and then a separately placed pliable optic secured within the outer haptic ring. Compression by the “elastic” lens capsule is meant to provide a compressive force to the central lens flexible optic. Other IOLs use a compressive force exerted on rigid haptics to compress a pliable optic against a separate fixed power posterior lens. These IOLs rely on the shape change of the posterior surface of the pliable optical element pressed against a fixed optical element or pressed against a relatively rigid posterior lens capsule to alter the dioptric power of the lens system. Other IOLs incorporate a skirt with a capsular contact ring. Such IOLs rely on compression exerted by the “elastic” lens capsule to impart a compressive force on a capsular contact ring and the mechanical design of this ring pulls radially about the equator of the IOL's flexible optic. Again, because these IOLs rely on retained capsular elasticity/pliability and because it is generally known that the lens capsule following cataract surgery becomes less pliable and more fibrotic, it is unlikely these lenses will retain accommodating/dis-accommodating ability. None of the shape changing accommodating IOLs described above mimic the natural human lens during accommodation or effectively account for the inevitable loss of capsular elasticity/pliability and progressive fibrosis and stiffening of the lens capsule.

### SUMMARY

The present disclosure relates to ophthalmic devices including accommodating intraocular lenses (accommodating IOLs). In an aspect, a method of manufacturing an integral fluid filled IOL comprising an anterior part and a posterior part is provided. The method can comprise mounting the anterior part of the IOL on a first nest, the anterior part defining a receptacle and having at least one haptic extending from an anterior face thereof. The method can further include introducing a pre-determined desired amount of an optical fluid or gel into the receptacle. The method can also include introducing a bonding agent into a trough of the posterior part of the IOL. The method can further comprise mounting the posterior part on a second nest. The method can further include applying force to draw the first nest towards the second nest or the second nest towards the first nest or placing the first and second nests in a vacuum sealed chamber and mechanically drawing the first and second nest together or placing the first and second nests in an inert gas chamber such that the posterior part and the anterior part are mechanically join together to form a fluid filled integral IOL. The method can then include removing the integral fluid filled IOL from the first and second nests.

### BRIEF DESCRIPTION OF THE DRAWINGS

FIG. 1 is a perspective cross-sectional view of a shape-changing optic of an IOL according to an aspect of the present disclosure.

FIG. 2 is a side view of a shape-changing optic of an IOL according to an aspect of the present disclosure.

FIG. 3 is a perspective view of the IOL including the shape changing optic of FIGS. 2 and 3 including a depiction of haptics according to an aspect of the present disclosure.

FIG. 4 is a perspective cross-sectional view of an IOL according to another aspect of the present disclosure.

FIG. 5 is a side view of an IOL according to an aspect of the present disclosure.

FIG. 6 is a side view of an IOL according to another aspect of the present disclosure.

FIG. 7 is a perspective cross-sectional view of a shape-changing optic of an IOL according to an aspect of the present disclosure.

FIG. 8 is a side cross-sectional view of a shape-changing optic of an IOL according to an aspect of the present disclosure.

FIG. 9 is a is a side cross-sectional view of a shape-changing optic of an IOL according to an aspect of the present disclosure.

FIG. 10 is a side view of an IOL according to another aspect of the present disclosure.

FIG. 11 is a side view of an IOL according to another aspect of the present disclosure.

FIG. 12 is a side view of an IOL according to another aspect of the present disclosure.

FIG. 13 is a side view of a shape-changing optic of an IOL according to an aspect of the present disclosure.

FIG. 14 is a side view of an IOL according to another aspect of the present disclosure.

FIG. 15 is a perspective cross-sectional view of the IOL of FIG. 14 according to an aspect of the present disclosure.

FIG. 16 is an exploded view of an IOL according to an aspect of the present disclosure.

FIG. 17 is a side cross-sectional view of an IOL according to an aspect of the present disclosure.

FIG. 18 is a partial cut-away perspective view of part of an IOL according to an aspect of the present disclosure.

FIG. 19 is a partial cut-away perspective view of part of an IOL according to an aspect of the present disclosure.

FIG. 20 is an exploded view of an IOL according to an aspect of the present disclosure.

FIG. 21 is a side cross-sectional view of an IOL according to an aspect of the present disclosure.

FIG. 22 is a side view of a first nest mounted on an anvil tool according to an example of an aspect of a method of manufacturing an IOL according to the present disclosure.

FIG. 23 is a side view of the first nest and anvil tool of FIG. 22 with an anterior part of IOL mounted on the first nest according to an example of an aspect of a method of manufacturing an IOL according to the present disclosure.

FIG. 24 is a side view of the assembly of FIG. 23 after fluid has been introduced into the receptacle/chamber of the anterior part of FIG. 23 to an example of an aspect of a method of manufacturing an IOL according to the present disclosure.

FIG. 25 is a side view of the assembly of FIG. 24 with a guide bushing installed over the first nest of FIG. 24 according to an example of an aspect of a method of manufacturing an IOL according to the present disclosure.

FIG. 26 is a side view of a bonding agent injector positioned within a trough of a posterior part of a IOL according to an example of an aspect of a method of manufacturing an IOL according to the present disclosure.

FIG. 27 is a close-up view of the injector positioned within the trough of the posterior part illustrated in FIG. 26 according to an example of an aspect of a method of manufacturing an IOL according to the present disclosure.

FIG. 28 is a close-up view illustrating bonding agent being introduced into the trough of the posterior part illustrated in FIG. 27 according to an example of an aspect of a method of manufacturing an IOL according to the present disclosure.

FIG. 29 is a side view of a posterior part of an IOL mounted in a second nest and piston tool according to an example of an aspect of a method of manufacturing an IOL according to the present disclosure.

FIG. 30 is a side and close-up view of a posterior part of an IOL mounted in a second nest having a retainer disposed therein and a piston tool according to an example of an aspect of a method of manufacturing an IOL according to the present disclosure.

FIG. 31 is a side view and a close-up view of the posterior part mounted in a second nest of FIG. 30 that, in turn, is mounted in a guide bushing and exposed to an applicator tool having a bonding agent deposited on a top surface thereof according to an example of an aspect of a method of manufacturing an IOL according to the present disclosure.

FIG. 32 is a side view and a close-up view of the assembly of FIG. 31 illustrating the bonding agent contacting the trough of the posterior part of the IOL according to an example of an aspect of a method of manufacturing an IOL according to the present disclosure.

FIG. 33 is a side view and a close-up view illustrating the alignment of the posterior part and the anterior part of the IOL according to an example of an aspect of a method of manufacturing an IOL according to the present disclosure.

FIG. 34 is a side view and close-up view of the assembly of FIG. 33 illustrating suction being applied to the assembly of FIG. 33 according to an example of an aspect of a method of manufacturing an IOL according to the present disclosure.

FIG. 35 is a side schematic illustration of an inert gas chamber in which the anterior part and the posterior part can be placed in to join both parts together according to an example of an aspect of a method of manufacturing an IOL according to the present disclosure/

FIG. 36 is a side view and a close-up view of an assembled fluid filled integral IOL according to an example of an aspect of a method of manufacturing an IOL according to the present disclosure.

### DETAILED DESCRIPTION

The present disclosure relates to an accommodating IOL. As used herein with respect to a described element, the terms “a,” “an,” and “the” include at least one or more of the described element(s) including combinations thereof unless otherwise indicated. Further, the terms “or” and “and” refer to “and/or” and combinations thereof unless otherwise indicated. By “substantially” is meant that the shape or configuration of the described element need not have the mathematically exact described shape or configuration of the described element but can have a shape or configuration that is recognizable by one skilled in the art as generally or approximately having the described shape or configuration of the described element. As used herein, the terms “anterior,” “posterior,” “superior,” “inferior,” “lateral,” and “medial” refer to the position of elements when a patient is in a standard anatomical position unless otherwise indicated. As used herein a “posterior face” is interchangeable with a “posterior optic” unless indicated otherwise. The terms “left,” “right,” “top” and “bottom” refer to the position of elements as they are depicted in the drawings and the terms “left” and “right” can be interchanged unless indicated otherwise. The terms “first,” “second,” etc. are used to distinguish one element from another and not used in a quantitative sense unless indicated otherwise. Thus, a “first” element described below could also be termed a “second” element. A component operably coupled to another component can have intervening components between the components so long as the IOL can perform the stated purpose. By “integral” or “integrated” is meant that the described components are fabricated as one piece or multiple pieces affixed during manufacturing or the described components are otherwise not separable using a normal amount of force without damaging the integrity (i.e. tearing) of either of the components. A normal amount of force is the amount of force a user would use to remove a component meant to be separated from another component without damaging either component. As used herein a “patient” includes a mammal such as a human being. All IOLs as described herein are used for medical purposes and are therefore sterile. Components of IOLs as described herein can be used with IOLs described herein as well as other IOLs. For example, an IOL as described herein can be placed anterior to an existing, previously placed IOL. IOLs include fixed power, multifocal, EDOF, diffractive and other variable focus lenses. Although the drawings show certain elements of an IOL in combination, it should be noted that such elements can be included in other embodiments or aspects illustrated in other drawings or otherwise described in the specification. In other words, each of the disclosed aspects and embodiments of the present disclosure may be considered individually or in combination with other aspects and embodiments of the disclosure including patent applications incorporated by reference herein.

Unlike shape changing accommodating IOLs described by way of background, IOLs are provided herein that can mimic the gradient elastic properties of a natural youthful human lens during accommodation and include a shape-changing optic where components of the optic change shape as the IOL transitions from an accommodated state to a dis-accommodated state and vice versa. Without wishing to be bound by a specific mechanism of action, it is considered by some that the lens capsules' “elasticity” controls and shapes the lens as a whole (the lens nucleus and cortex). On this basis, the lens contents are considered pliable.

However, the volume of the lens contents compared to the thickness and known modulus of elasticity of the lens capsule predicts that the lens capsule cannot solely control and alter the shape of the lens nucleus and cortex. Finite element analysis (FEA) predicts that radial tension about the equatorial region of a lens capsule filled with a soft pliable solid or liquid does not result in significant shape change to either the anterior or posterior surface of the lens compared to what is known to occur with the natural youthful human lens. Providing radial tension directed specifically to at least the anterior face of an accommodating IOL; having that tension directed at points anterior to the equator of the IOL; the anterior face of the IOL being more resistant to deformational change than the content(s) of a chamber underlying the anterior face; the anterior face demonstrating elastic properties in so much as the anterior face deforms when a force is applied to the anterior face and the anterior face will return to its original shape with the removal of the force, results in a greater amount of anterior face shape change and therefore accommodating dioptric power change than can be achieved with a similar force applied at points at or more near the equator of the IOL (e.g. equatorial). In addition, a force applied to the anterior face at points anterior to the equator of the IOL requires less diameter change of the anterior face per diopter of power change of the IOL compared to a similar force applied at points at or more near the equator of the IOL thereby allowing the anterior face of the IOL to shape change even with very small amounts of anterior face diameter change when going from an accommodated state, a dis-accommodated state, and states in between.

In particular, in an aspect, an IOL comprising a shape changing optic that can assume an accommodated state, a dis-accommodated state, and states therebetween is provided. Components of the shape-changing optic can be deformable such that radially directed ocular compression force(s) or tensile force(s) applied to the optic caused by ciliary muscle contraction or relaxation causes one or more components of the optic to change shape and allows the optic to change dioptric power. None of the described IOLs above apply a radially outward tensile force that is directly transferred to the anterior surface at point(s) anterior to the equator of the optic

As such, components of a shape-changing optic can deform or change shape when a force is applied. If a component is less resistant to deformational change than another component, the former component is more likely to, or to a greater degree, deform for a given amount of applied or removed force than the latter component. A component is more resistant to deformational change than another component, if the former component is less likely to, or to a lesser degree, deform for a given amount of applied or removed force than the latter component. It is understood that for any given component resistant to deformational change, the force applied/removed to such component does not exceed the force that results in breakage of the component such that it is no longer useful for its therapeutic purpose.

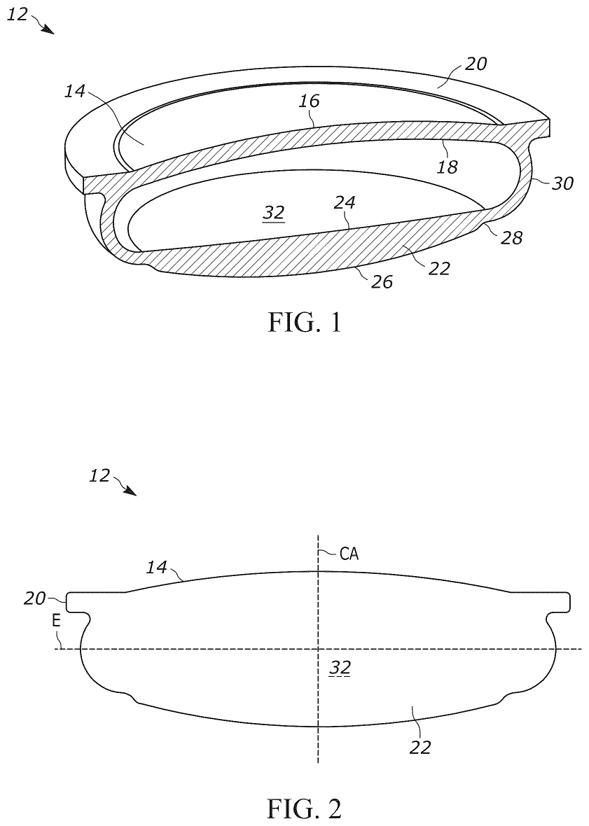

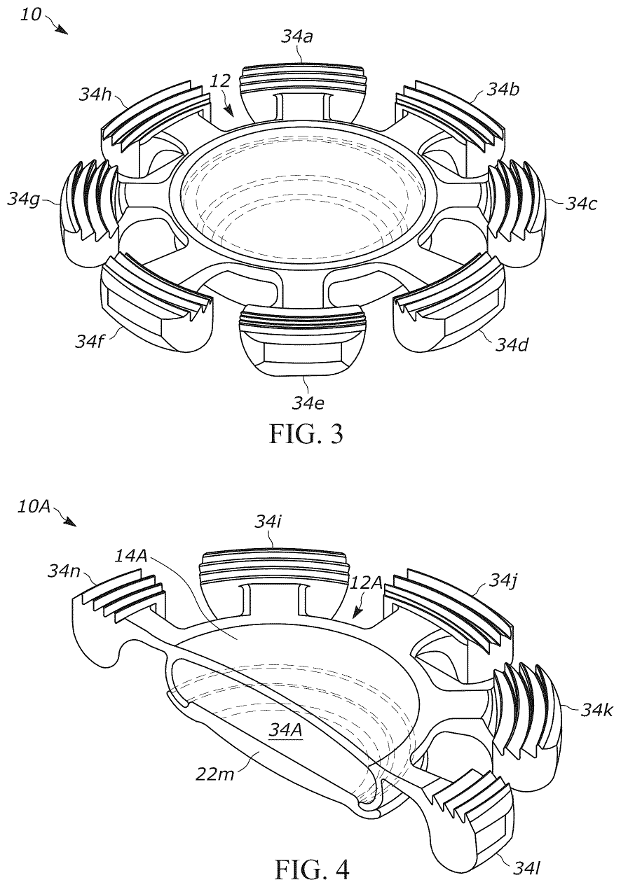

FIG. 2 depicts a central or optical axis CA extending in an anterior-posterior direction and an equator E extending in a plane substantially perpendicular to the central axis. The equator is an imaginary line drawn around the circumference of a lens perpendicular to the optical axis, equally distant from the anterior face of the lens and the posterior face of the lens, dividing the lens into an anterior half and a posterior half. Referring to FIGS. 1-3, a shape-changing optic 12 of an IOL 10 can comprise an elastic anterior face 14 located anterior to equator E. Anterior face 14 can have an anterior surface 16, a posterior surface 18 and a periphery 20. Shape-changing optic 12 can also comprise a posterior face 22 having an anterior surface 24, a posterior surface 26, and a periphery 28. Shape-changing optic 12 can further include an elastic side wall 30 extending across equator E and extending from anterior face 14 to posterior face 22. A chamber 32 can be located between anterior face 14 and posterior face 22 and can house material or contents as described in more detail below. Components of the shape-changing optic can be made to be more or less resistant to deformational change by altering the thickness of the component, the type of material from which the component is fabricated, or by altering the chemical/material properties of the component material itself for example. With reference to FIG. 3, IOL 10 can further comprise at a plurality of haptics 34 extending from the periphery of the anterior face. A plurality of haptics can also extend from the periphery of the posterior face, or the periphery of both the anterior face and the posterior face as described below.

Regarding specific components of an IOL, the anterior face, as stated above, can have elastic properties. Elastic properties can allow for the anterior face to change shape with an applied force, but also to return to its original configuration when the force is removed. It is beneficial that the anterior face be more resistant to deformational change (e.g. less pliable, firmer) than the contents or material contained within the chamber because when an outward radial force is applied to the anterior face, the contents of the chamber can more easily deform to allow flattening of the anterior face. Exemplary fabrication materials for the anterior face include silicone, an acrylic (hydrophobic or hydrophilic) polymer, polymethylmethalcryalate (PMMA), silastic, collamer, a suitable optical thermoplastic polymer, another suitable optical material, and suitable combinations thereof. The anterior face can comprise a lens with a variety of optical properties, such as, for example, a spherical, aspheric, toric, toroidal, multifocal, diffractive, extended depth of focus, or combinations thereof.

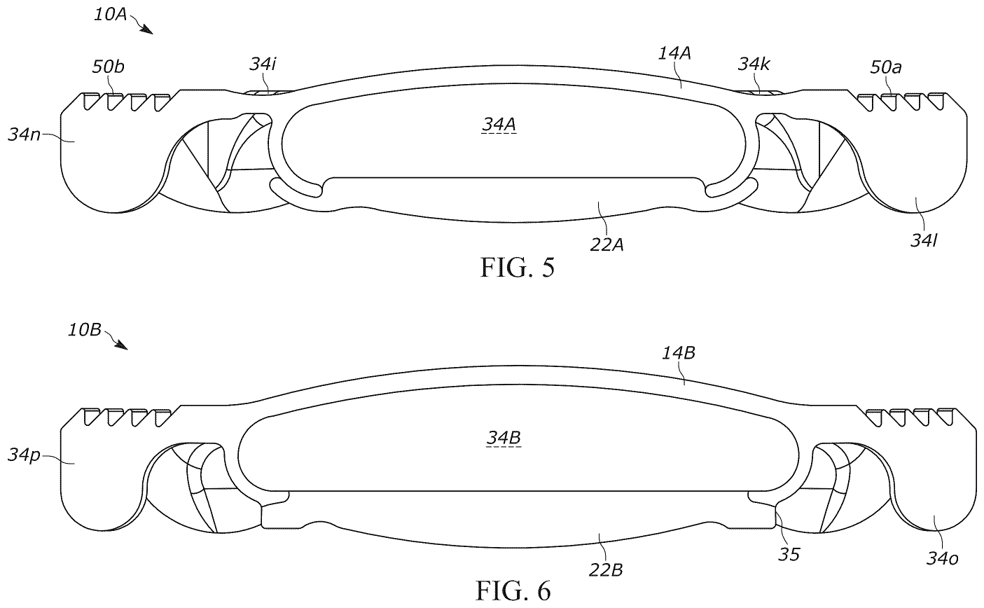

Regarding the posterior face of the shape-changing optic, the posterior face can be more resistant to deformational change than the anterior face or the contents contained within the chamber of the shape-changing optic. The posterior face need not have the ability to change shape. When implanted and in certain aspects, the posterior face can rest against the posterior capsule and the vitreous substance and it may not be desirable to have those less predictable forces altering the power of the optic. Furthermore, having a posterior face that is more resistant to deformational change than the anterior face or the contents of the chamber of the shape-changing optic can allow the posterior face optic to have a relatively more fixed power posterior lens permitting the incorporation of beneficial optical properties. In addition, a posterior face more resistant to deformational change can allow the contents of the chamber to reshape the side wall(s) when the anterior face changes shape in response to a force. The posterior face can be part of a one piece fluid filled integral IOL 10 as depicted in FIG. 1-3 or can be a two-piece fluid filled integral IOL 10A as illustrated in FIG. 4-6. In certain aspects, the posterior face is elastic. Exemplary fabrication materials for the posterior face include silicone, an acrylic (hydrophobic or hydrophilic) polymer, polymethylmethalcryalate (PMMA), silastic, collamer, a suitable optical thermoplastic polymer, another suitable optical material, or suitable combinations thereof. The posterior face can comprise a lens with a variety of optical properties, such as, for example, a spherical, aspheric, toric, toroidal, multifocal, diffractive, extended depth of focus, or combinations thereof. As illustrated in FIG. 6, an IOL 10B can comprise a shape-changing optic where the posterior face 22B has a squared peripheral edge 35 to reduce posterior capsular opacification, by inhibiting, for example, peripheral lens epithelial cells from migrating across the posterior face.

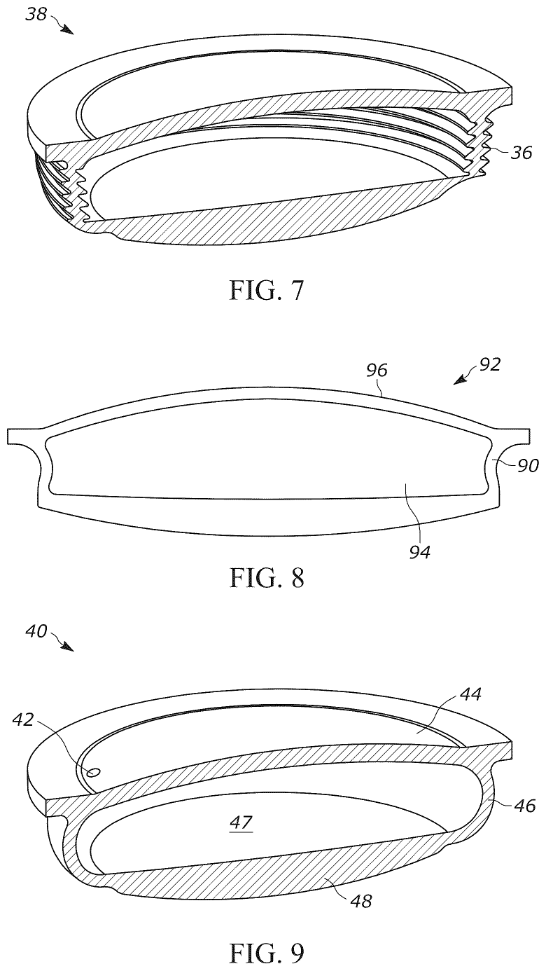

Regarding the side wall, as stated above, the side wall can have elastic properties. In certain aspects, the side wall can be fabricated from a material that is equal to or less resistant to deformational change than the anterior face. Such features can allow for the contents contained within the chamber to expand the area of the side wall to allow the volume of the contents of the chamber to remain the same when the anterior surface is flattened. Having the side wall deform can facilitate and allow for a greater amount of shape change to the anterior face of the shape-changing optic. Exemplary fabrication materials for the side wall include silicone, an acrylic (hydrophobic or hydrophilic) polymer, polymethylmethalcryalate (PMMA), silastic, collamer, a suitable optical thermoplastic polymer, another suitable material, or a suitable combination thereof. The side wall can also be equal to or less resistant to deformational change than the anterior face or the posterior face by being thinner than the anterior face or the posterior face. Alternatively, or in addition, the side wall 36 of a shape-changing optic 38 can be equal to or less resistant to deformational change by having a bellowed configuration as illustrated in FIG. 7. The bellows can be horizontally or vertically oriented or have other orientations to allow for peripheral side wall expansion or contraction. As illustrated in FIG. 8, the side wall 90 of a shape changing optic 92 can have a plano, concave, convex, or other configuration to facilitate displacement of the contents of chamber 94 against side wall 90 when anterior face 96 is flattened.

Regarding the chamber, the chamber can be defined by the posterior surface of the anterior face, the anterior surface of the posterior face, and an inner surface of the side wall. The interior contents or material of the chamber can comprise a soft solid, a gel, a viscoelastic material, a flowable fluid, or a gas, or other suitable material. Exemplary materials that can be contained within the interior of the chamber include a soft silicone, or other soft material subject to deformational change, air or other gas, silicone oil (of various refractive indices), an aqueous solution of saline or hyaluronic acid, a viscoelastic polymer, polyphenyl ether, or other optical fluid, solid or gases, or suitable combinations thereof. The chamber can have an internal layer or coating, such as a parylene coating for example, to seal the contents of the chamber from the anterior face, the side wall and/or the posterior face. The chamber can be pre-loaded (e.g. by a manufacturer) with a suitable material. Alternatively, the chamber can be loaded with a suitable material by a clinician. For example, and with reference to FIG. 9, a shape-changing optic 40 of an IOL can define at least one port 42 (enlarged in FIG. 9 for purposes of clarity) sized and dimensioned to receive a needle or catheter, the needle or catheter being sized and dimensioned to deliver a fluid, gel, or gas to the chamber and/or to exchange fluid with a different material or a material having a different refractive index, for example. Although FIG. 9 illustrates the port defined by anterior face 44, the port can be defined by the side wall 46 or the posterior face 48 of the shape-changing optic. Having a port can allow a user to add or remove substance from chamber 47 to adjust the optical power of the lens. For example, by adding additional substance to the chamber, the volume of the substance can increase in the chamber resulting in an increase in the surface(s) curvature and the overall power of the lens and removing substance can decrease the volume of the substance in the chamber resulting in a decrease in the surface(s) curvature and the overall power of the lens. Also, by exchanging the substance for one with a different refractive index, the overall dioptric power and the range of accommodation of the IOL can be increased or decreased.

Regarding the plurality of haptics of the IOL, such haptics are the portion of the IOL that are configured to interact with the lens capsule, the lens zonules, the ciliary muscle, or other parts of a patient's eye. The plurality of haptics can be molded, shaped into, integral with, or otherwise extend from the shape-changing optic of an IOL. The plurality of haptics can be elastic but can be more resistant to deformational change than the anterior face. An advantage to this is that the haptics can be firmer to provide a linear, radially directed force from the haptic that is directly transferred to the periphery of the anterior face. Without wishing to be bound by any particular mechanism of action, if the haptics were less resistant to deformational change than the anterior face, the radial tension could result in stretching of the haptics and less tension on the periphery of the anterior face. Thus, the anterior face may not shape change as much for a given force applied to the haptics. Exemplary fabrication materials for the haptics include silicone, an acrylic (hydrophobic or hydrophilic) polymer, polymethylmethalcryalate (PMMA), silastic, collamer, a suitable optical thermoplastic polymer, another suitable material, or suitable combinations thereof.

FIGS. 3 to 6 illustrate an IOL where a plurality of haptics extends from the anterior face of a shape-changing optic. The shape-changing optic can change shape in response to an ocular force, specifically a force generated by the contraction or relaxation of the ciliary muscle of the patient's eye. The plurality of haptics, interacting with the lens capsule, can apply radial outward tension to the anterior face when the ciliary muscle relaxes and radial outward tension is placed on the lens capsule via the lens zonules. The plurality of haptics can be elastic but can be equal to or more resistant to deformational change than the anterior face.

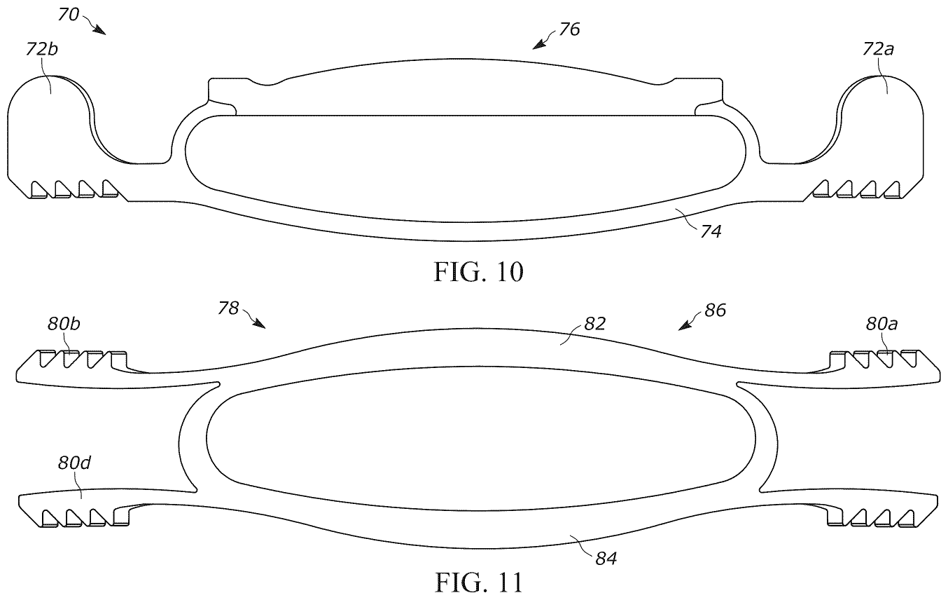

FIG. 3 illustrates an aspect where a plurality of haptics extends circumferentially from the shape-changing optic. When implanted and when the ciliary muscles of a patient's eye relaxes (such as when the eye is in a dis-accommodated state), the ciliary muscles apply tensile force to the plurality of haptics (via the lens capsule with lens zonule attachments between the lens capsule and the ciliary muscles, for example). The plurality of haptics, in turn, can apply tensile force to the periphery of the anterior face at each site (referred to herein as an “extension site”) where a respective haptic extends from the periphery of the anterior face. By having a plurality of haptics as depicted in FIGS. 3 and 4, the net result can be that the anterior face can be pulled radially outward substantially perpendicular to the optical axis from several extension sites (such as, for example, eight extension sites as illustrated in FIG. 3) and functionally result in relatively symmetric radial tension placed on the periphery of the anterior face of the shape-changing optic. Referring to FIG. 10, in certain aspects, an IOL 70 includes a plurality of haptics 72 extending from posterior face 74 of shape-changing optic 76. By having the force applied to the posterior face, a change in shape of the optic can be achieved independent of or in combination with a force applied to the anterior face. Referring to FIG. 11, in other aspects, an IOL 78 includes a plurality of haptics 80 extending from anterior face 82 and posterior face 84 of shape-changing optic 86. If a force is applied to both the posterior and the anterior faces, the total dioptric power change of the IOL for a given force can be increased. In other words, if a force is applied to both the anterior and posterior face, shape change can be obtained to both surfaces and thereby increase overall accommodation.

The plurality of haptics can engage the inner surface of the lens capsule or the outer surface of the lens capsule. Referring to FIGS. 3-6, the peripheral portion of the plurality of haptic 34 can comprise fixation members such as ridges 50 or other alternating raised and recessed areas as illustrated in FIGS. 3-6 configured to engage an inner surface of a lens capsule. For example, ridges 50 or alternating raised and recessed areas, etc. can interact with the lens capsule to stabilize the haptics within the lens capsule. Alternating raised and recessed areas can also allow the haptics to interact and fixate into the lens capsule. Such an aspect can allow IOL placement within the capsular bag, while still allowing translation of tension/relaxation of the lens capsule (via the lens zonules and ciliary body) during accommodation/dis-accommodation of the lens. Current haptic designs do not allow the haptics to be positioned into the lens capsule while fixating the haptics to allow tension/relaxation on the lens capsule to translate forces into the haptics. Current haptic designs are smooth and allow the capsule and haptic to glide past each other, which does not allow the translation of forces placed on the peripheral lens capsule (via the zonules and ciliary muscle). When placing the IOL inside the capsular bag, the ridges/dimples or other alternating areas of raised and recessed areas on the haptics fixate the lateral portions of the haptics to the inside periphery area/portion of the lens capsule. The alternating areas of raised and recessed areas then provide resistance to the lens capsule gliding radially outward over the peripheral haptic and facilitates the forces from the ciliary body, zonules, and lens capsule into a force on the haptics.

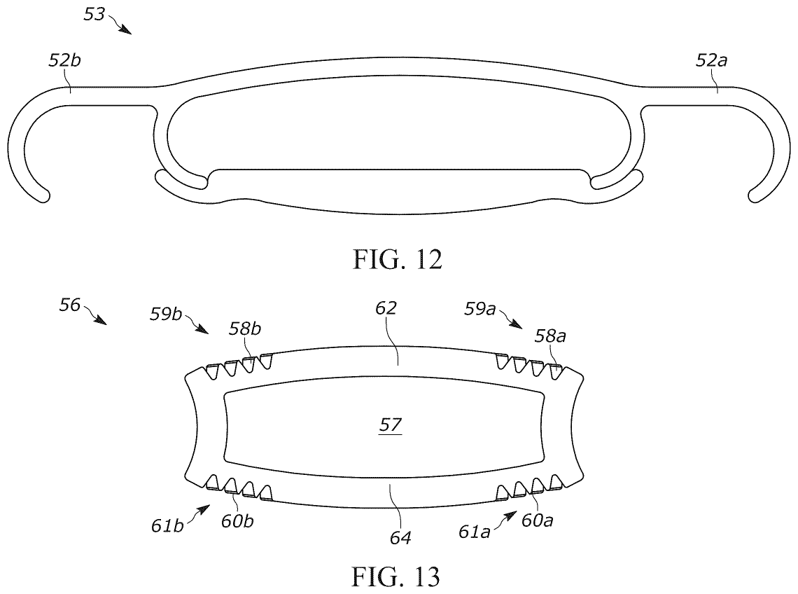

Regarding the plurality of haptics engaging the outer surface of the lens capsule, when an IOL is placed anterior to an existing, previously implanted IOL, or when placed anterior to the lens capsule, the haptics can engage the outer surface of the lens capsule. Referring to FIG. 12, the peripheral portion of haptic 52 of an IOL 53 can comprise a hook-shaped/substantially J-shaped configuration to engage or curve around an outer surface of a lens capsule. The peripheral end of the peripheral portion can be an atraumatic end so that it does not damage zonules or the lens capsule. Each of the plurality of haptics can comprise a hook. Hook or substantially J-shaped haptics can allow an IOL to use the force translated from the ciliary muscle to the lens capsule, via the lens zonules, without requiring placement of haptics against elements of the ciliary muscle. Such an embodiment can avoid known potential complications of haptics placed against the ciliary muscle, such as uveitis, glaucoma, and bleeding (e.g. hyphema). Such an embodiment can be implemented in patients that have an already implanted IOL or patients that do not have an already implanted IOL.

Referring to FIG. 13, the shape-changing optic itself can define ridges to engage the inner surface of a lens capsule. For example, the shape-changing optic 56 can include an expandable chamber 57, that has an integrated haptic with ridges 58 and 60 on periphery 59 of the anterior face 62 and/or the periphery 61 of the posterior face 64. When tension is placed on the lens capsule by the zonules (e.g. when the ciliary muscle relaxes), the force can be translated (by the ridges engaging the capsule) specifically to the anterior and posterior faces and not just translation of a general force to the entire lens. The anterior face, the posterior face, and/or the side walls can be more resistant to deformational change than the contents of the chamber. This configuration can allow forces from the lens capsule to provide radial tension to the haptics and thus to the anterior face, the anterior face being anterior to the equator of the lens; and/or to provide radial tension to the posterior face, the posterior face being posterior to the equator of the lens. The side walls can be configured to allow for the material in the chamber displaced by the flattening of the anterior face and/or the posterior face to expand into the area of the side wall, thereby allowing the volume of material within the chamber to remain the same.

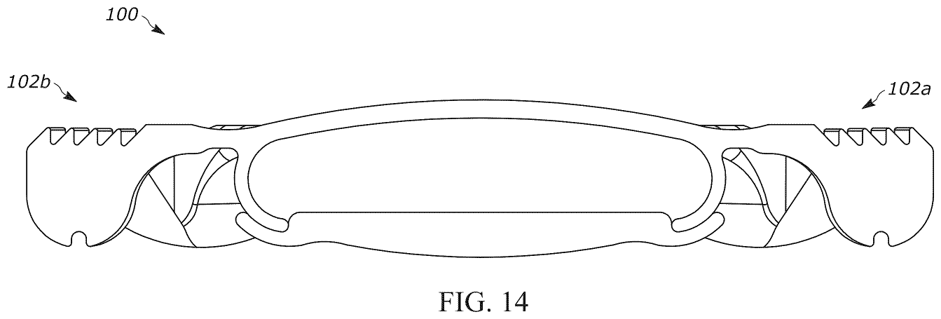

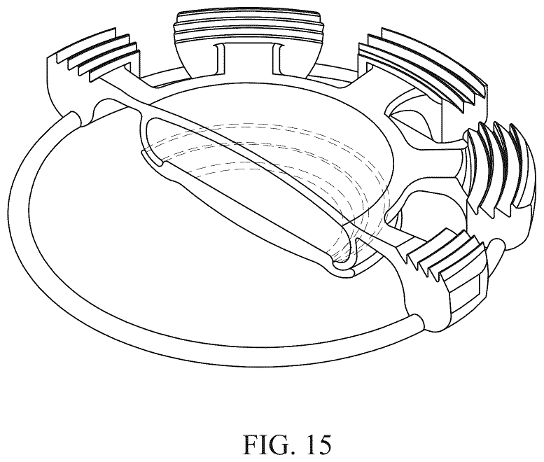

Referring to FIGS. 14 and 15, in certain aspects, an IOL 100 is provided where the bottom of haptic 102 defines a recess 104. Such a recess can accommodate a stabilizing ring 106, for example, to keep the haptics from holding inwards with lens capsule fibrosis. Stabilizing ring can be fabricated from any of the materials described above with respect to the anterior and/or posterior faces of the shape changing optic.

In certain aspects, the haptics can contain a therapeutic agent. Non-limiting examples of therapeutic agents include an intraocular steroid, an antibiotic to mitigate post-operative inflammation/infection such that tear drops may not be necessary, a therapeutic agent for improving glaucoma or macular degeneration, or combinations thereof. For example and with respect to chronic conditions such as glaucoma or macular degeneration, the therapeutic agent can be placed in the recessed areas of the haptics (in embodiments having such recessed areas) for long-term of sustained release of the therapeutic agent.

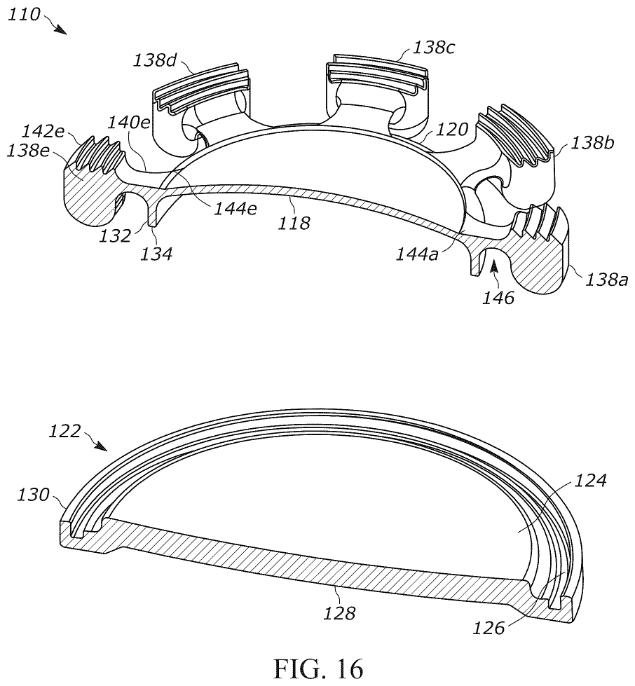

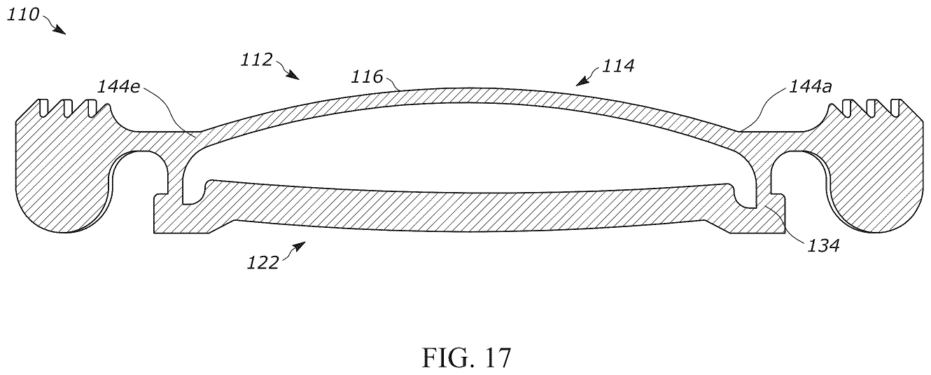

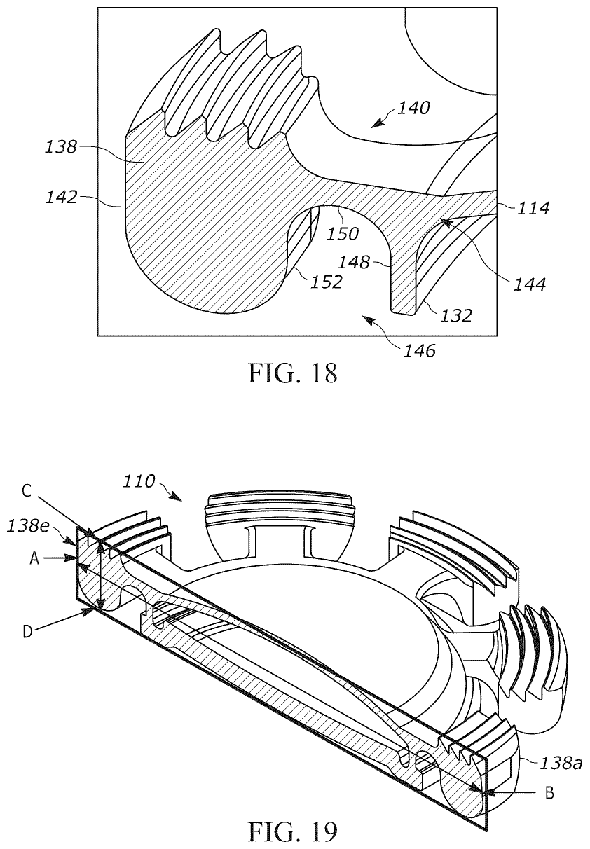

Referring to FIGS. 16-18, in an aspect, an IOL 110 is provided comprising a shape-changing optic 112 configured for placement in or adjacent to a lens capsule. IOL 110 has an optical axis extending in an anterior-posterior direction, an equator extending in a plane substantially perpendicular to the optical axis, an accommodated state, a dis-accommodated state, and states therebetween. The optical axis and equator are described above with reference to FIG. 2, for example. IOL 110 comprises an elastic anterior face 114 located anterior to the equator. Anterior face 114 has an anterior surface 116, a posterior surface 118, and a periphery 120. IOL 110 further includes a posterior face 122 located posterior to the equator. Posterior face 122 has an anterior surface 124 defining an annular channel 126, a posterior surface 128, and a periphery 130. IOL 110 further includes an clastic side wall 132 extending across the equator and extending from anterior face 114 to posterior face 122. Side wall 132 further includes a posterior edge 134 complimentary to and non-releasably bonded to annular channel 126 of posterior face 122.

Such an engagement between the posterior edge of the side wall and the annular channel of the posterior face allows for a stronger bond between the anterior face and the posterior face of the optic. In this aspect, when injection molding, the IOL is manufactured in two parts. One part includes the anterior face, the plurality of haptics, and the side wall. The posterior face is molded separately and includes the posterior surface of the posterior face and the annular channel defined by the anterior surface of the posterior face. The annular channel facilitates attaching the posterior face of the IOL to the anterior face. By having the annular channel, the posterior portion (including the posterior edge) of the side wall can fit into annular channel. The annular channel can be partially filled with uncured silicone, silastic material, acrylic, other material, or with a suitable bonding agent for example. The anterior face and the posterior face can then be permanently bonded by curing the silicone or allowing bonding agents to “set,” thus forming a sealed chamber.

An advantage of this configuration is that molding the anterior face and the posterior face separately allows the optical surfaces to be highly polished. Molds can be polished to very high optical finish such as, for example, SPI standard A-1 (6000 grit). When components of the IOL are released from the molds, the anterior and posterior surfaces of the anterior face, and the anterior and posterior surfaces of the posterior face can all be highly smooth optical finishes. Because of this, the fluid, for example, filling the chamber does not need to have a refractive index that matches the components of the IOL that define the chamber. For example, the refractive index of silicone, which can be a material that defines the chamber, is approximately 1.41 and silicone oil, which can be material inside the chamber could be 1.51.

Such a configuration of an IOL is more advantageous than configurations of IOLs that are manufactured using blown in molding whereby a single mold is formed and the material is injected (blown in) to adhere to the inner wall of the mold forming the anterior part and posterior part and a chamber as a single unit. This allows for the anterior surface of the anterior part and the posterior surface of the posterior part to have a high-grade optical finish, but the posterior surface of the anterior face and the anterior surface of the posterior face may not have optically smooth finishes (in other words the inside wall which form the chamber will not be optically smooth). When this is the case, the optical fluid within the chamber may have a refractive index matched to avoid/limit optical phenomena (such as, e.g. scatter and reflection) which occurs when light passes through the interface of two materials with differing refractive indices.

IOL 110 further includes a chamber 136 located between anterior face 114 and posterior face 122 and containing a material as described above. Anterior face 114 is more resistant to deformational change than the material within the chamber. IOL 110 also includes a plurality of haptics 138 each having a medial portion 140 and a lateral portion 142. The medial portion 140 of each of the haptics extends from and is connected (e.g. directly) to a respective extension site 144 of the periphery 120 of anterior face 114. In addition or alternatively, the IOL can also include an arch 146 located at the extension site 144 or the junction where each of the plurality of haptics extends from and is connected to the anterior face 114 of the optic. In particular, for any given arch 146, the arch is defined by the outer surface 148 of side wall 132, the posterior face 150 of the medial portion 140 of the haptic 138, and the inner surface 152 of the medial portion 140 the haptic 138. Such arches can stabilize the haptics at the extension sites and minimize flexion of the haptics at this junction or extension site.

In particular, the arches can prevent/limit rotation of a haptic at the extension site of the anterior face. This facilitates the haptics extending radially outward allowing the periphery of the haptics to engage the peripheral lens capsule. By maintaining the haptics in a radially oriented fashion, the transfer of radial force by the ciliary muscle/zonules/lens capsule/haptics results in a radial force applied to the anterior face and limits flexion/rotation of the medial portion of the haptics. The arches are configured to prevent haptic rotation yet produce only minimal radial force to the side wall. This can be achieved with the arch having the greatest width at the location where the medial portion of the haptic attaches to the anterior face (the extension site).

The arch defined by the medial portion of the haptic also can facilitate the peripheral portion of the haptic maintaining correct orientation. This allows for optimal fit of the peripheral portion of the haptic into the peripheral lens capsule. The arch can also prevent rotation/flexion of the peripheral portion of the haptic where it joins with the flat portion of the haptic.

Further regarding the haptics, in certain aspects, each of the plurality of haptics can be non-rotatable in response to axial compression along the optical axis on the shape-changing optic. In certain aspects, each of the haptics has a peripheral portion having a posterior face and an anterior face, with the posterior face being curved. In other aspects, the medial portion of each of the plurality of haptics medial portion extends from and is connected to the periphery of the anterior face such that the plurality of haptics changes the shape of the anterior face via application of radial tension to the periphery of the anterior face in a direction perpendicular to the optical axis. For example, the shape change of the anterior face is not via compressive forces along the optical axis on the shape-changing optic

Referring to FIG. 19, the IOL can be configured to have a relaxed configuration on the accommodated state. The IOL can be configured such that the haptics are fixated to the lens capsule. As stated above, the zonules attach the lens capsule to the ciliary muscle. When the ciliary muscle relaxes, a force is applied to the lens zonules and this force is translated to the lens capsule and thus to the lens haptics. This radial outward force pulls on the lens haptics which translates the force to the anterior face of the optic, thus decreasing the dioptric power of the optic. If the diameter (A-B) of the IOL were too large or the haptics were too thick (distance C-D) and stretched the lens capsule, increasing the diameter of the lens capsule, the zonules would be relatively slack with relation of the ciliary muscle and the lens capsule. As a result, the zonules may not translate the most optimum force to the lens capsule, and to the haptic arms and therefore the IOL may not change optical power as desired

If the diameter (A-B) of the IOL were too small or the haptics were too thin (distance C-D), the IOL may not be stable within the lens capsule and may not maintain a centered position within the lens capsule. To allow for natural variation in lens diameter and thickness, the width of the IOL (distance A-B) or the Anterior-Posterior distance of the haptic (distance C-D), or both, may be varied such that the calculated circumference IOL=will be between approximately 21.0 mm (for eyes that have smaller lenses) and approximately 27.00 mm (for eyes with larger lenses). This can allow the IOL to function properly for eyes of different sized lenses. This can be achieved by varying the diameter measured from points A-B, or by varying the A-P width of the end of the haptic from points C-D.

In another aspect, the IOL can have a circumference calculated as follows: 2× distance (A-B))+2× distance (C-D). Distance A-B is the distance from the most peripheral part of the peripheral surface of one haptic (e.g. 138c) (at point A) to the corresponding point on the opposing haptic (e.g. 138a) (at point B). Distance C-D is the distance from the most anterior part of a haptic (e.g. 138a) (point C) to the most posterior part of the same haptic (e.g. 138a) (point D).

The anterior-posterior dimension of each of the haptics can be at least 50% or more (even greater than 100%) than the anterior posterior dimension of the IOL. This provides separation of the anterior and posterior capsule in the periphery and facilitates an advantageous mechanical relationship between the IOL, the haptics, the zonules, and the ciliary muscle.

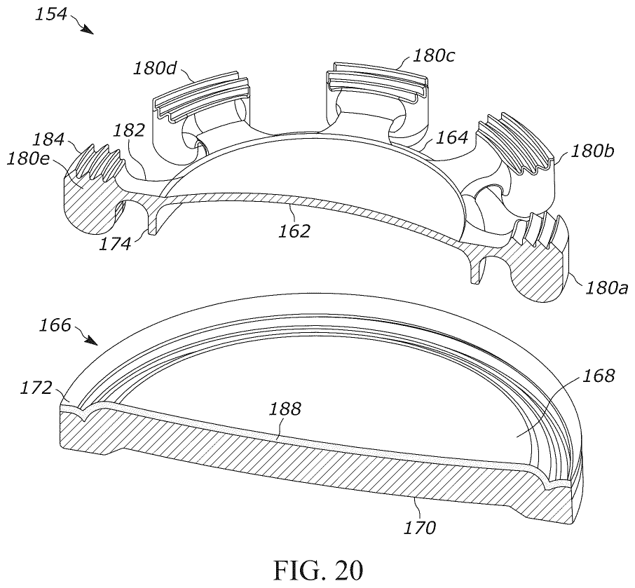

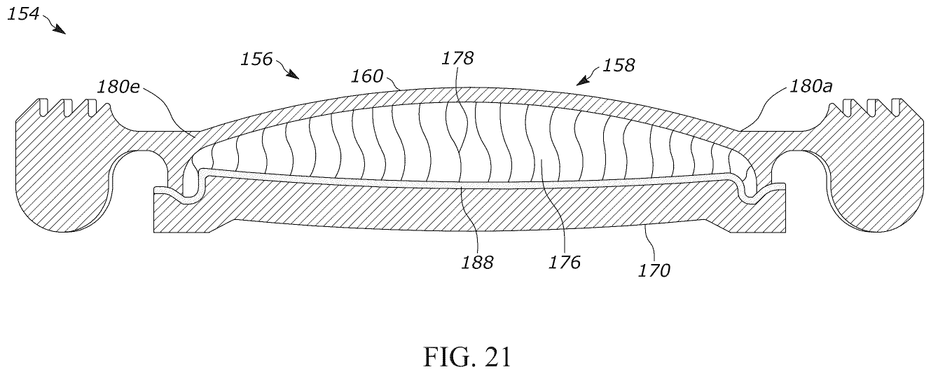

FIGS. 20 and 21 illustrate another aspect of the present disclosure directed to an accommodating IOL 154 where the refractive power of the IOL can be modified after implantation in the patient's eye. IOL 154 can comprise shape-changing optic 156 configured for placement in or adjacent to a lens capsule. The IOL has an optical axis extending in an anterior-posterior direction, an equator extending in a plane substantially perpendicular to the optical axis, an accommodated state, a dis-accommodated state, and states therebetween. IOL 154 can comprise clastic anterior face 158 located anterior to the equator and posterior optic 166 located posterior to the equator. Anterior face 158 can have anterior surface 160, posterior surface 162, and periphery 164. Posterior optic 166 can have anterior surface 168, posterior surface 170, and periphery 172. Elastic side wall 174 can extend across the equator and extend from anterior face 158 to posterior optic 166. Although the elastic side wall is depicted as having a posterior edge complimentary to and non-releasably bonded to an annular channel of the posterior optic, the elastic side wall and posterior optic need not necessarily have these features. Chamber 176 can be located between the anterior face and the posterior optic and can contain fluid 178 which can be, for example, a liquid or a gel. Anterior face 158 can be more resistant to deformational change than fluid 178. A plurality of haptics 180 each having medial portion 182 and lateral portion 184 can extend from and be connected to periphery 164 of anterior face 158.

Posterior optic 166 can include a refractive power adjustment material comprising silicone, for example, that allows modification of the refractive power of the IOL when exposed to a light source such as, for example, ultraviolet light. Such an optic can permit refractive power adjustment of an accommodating lens weeks or months after surgical implantation in the eye. As such, a surgeon can fine tune the refractive power of an accommodating lens for a specific patient. Non-limiting examples of materials that can be included in the posterior refractive power adjustment optic are described in U.S. Pat. Nos. 6,450,642, 6,851,804, 7,074,840 and 7,281,795, which are incorporated by reference in their entirety. Coating 188 can be disposed between posterior refractive power adjustment optic 166 and chamber 176. In certain aspects, the coating is disposed on anterior surface 168 of the posterior refractive power adjustment optic. Coating 188 can comprise a material that prevents entry (e.g. penetration, absorption, leaching) of fluid 178 in chamber 176 into refractive power adjustment optic 166 such that the fluid does not interfere with the ability of the posterior optic to change refractive power upon exposure to a light source. For example, the coating can comprise parylene, acrylic, a nanotechnology barrier, or a liquid conformational coating. In certain aspects, the fluid in chamber 176 can comprise a material that does not leach into the posterior refractive power adjustment optic rendering the coating optional or unnecessary. IOL 154 can optionally be combined with other aspects of an IOL(s) as disclosed above.

In another aspect, a method of manufacturing a fluid filled IOL is provided such that a precise amount of fluid can be introduced into the chamber or receptacle of the IOL and the anterior and posterior parts of the IOL can be precisely aligned with each other. In particular, a method of manufacturing a fluid filled IOL comprising an anterior part and a posterior part can comprise mounting the anterior part of the IOL on a first nest. The anterior part can comprise a receptacle defined by an anterior face and a side wall extending from the anterior face and can have at least one haptic extending from the anterior face. The method can further comprise introducing a pre-determined desired amount of a fluid, such as an optical fluid, gel, or oil into the receptacle. Such a pre-determined desired amount of fluid is the precise amount of fluid desired to be disposed within the IOL to provide the necessary functionality of the fluid in the integral fluid filled IOL post-manufacturing. The method can further comprise introducing a bonding agent into a trough, which can have an annular channel as disclosed above, of the posterior part of the IOL and mounting the posterior part on a second nest. The bonding agent can be an adhesive, a sealant or other suitable bonding agent. Mounting the anterior part and the posterior part of the IOL into the respective first and second nests (as well as introducing the fluid into the receptacle of the anterior part and introducing the bonding agent into the trough of the posterior part of the IOL) can be done in any suitable order. After the anterior and posterior parts have been mounted on their respective nests, fluid has been added to the receptacle of the anterior part, and a bonding agent has been applied to the trough of the posterior part, force such as suction/vacuum or mechanical compression for example, can be applied to draw the second nest toward the first nest (or vice versa) to seal the posterior part to the anterior part to form a fluid filled integral IOL. Alternatively, the anterior part and posterior part can be assembled in vacuum chamber. If necessary, heat may be applied prior to or after removing the IOL from the assembly. Alternatively, the entire assembly may be placed in a heat chamber to cure the bonding agent. The fluid filled integral IOL then can be removed from the respective nests. The anterior part, the posterior part and the bonding agent can be fabricated from the same base fabrication material to form a one-piece single integral IOL after manufacturing. For example, the posterior part and anterior part can be fabricated from silicone and the bonding agent can be a silicone material. Other materials could also be used (including those disclosed above). In the case where the bonding agent and the anterior part and the posterior part are fabricated from the same base fabrication material, such as silicone, once the sealant cures, the assembled IOL becomes a single piece integral IOL with the silicone oil in the receptacle/chamber.

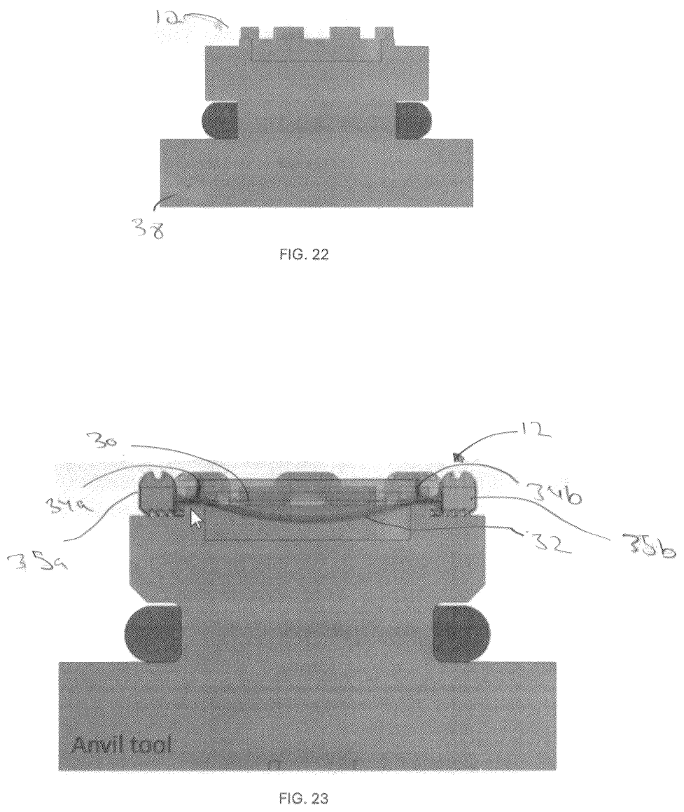

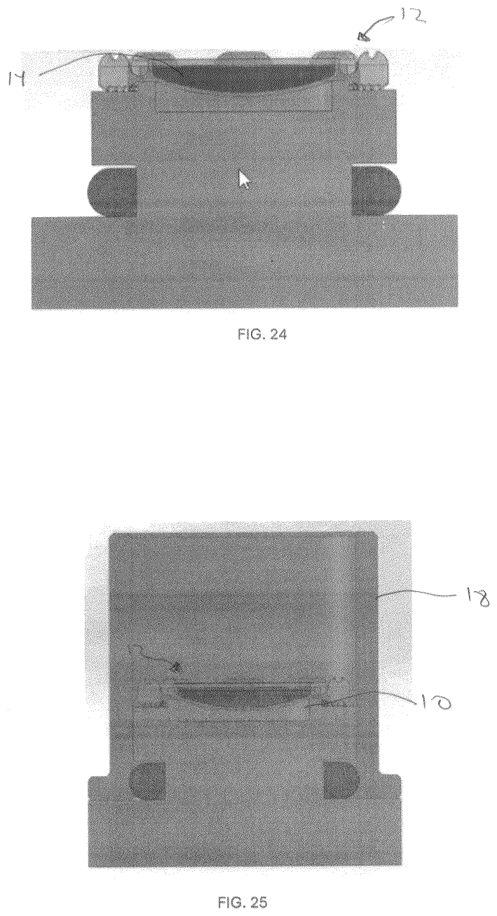

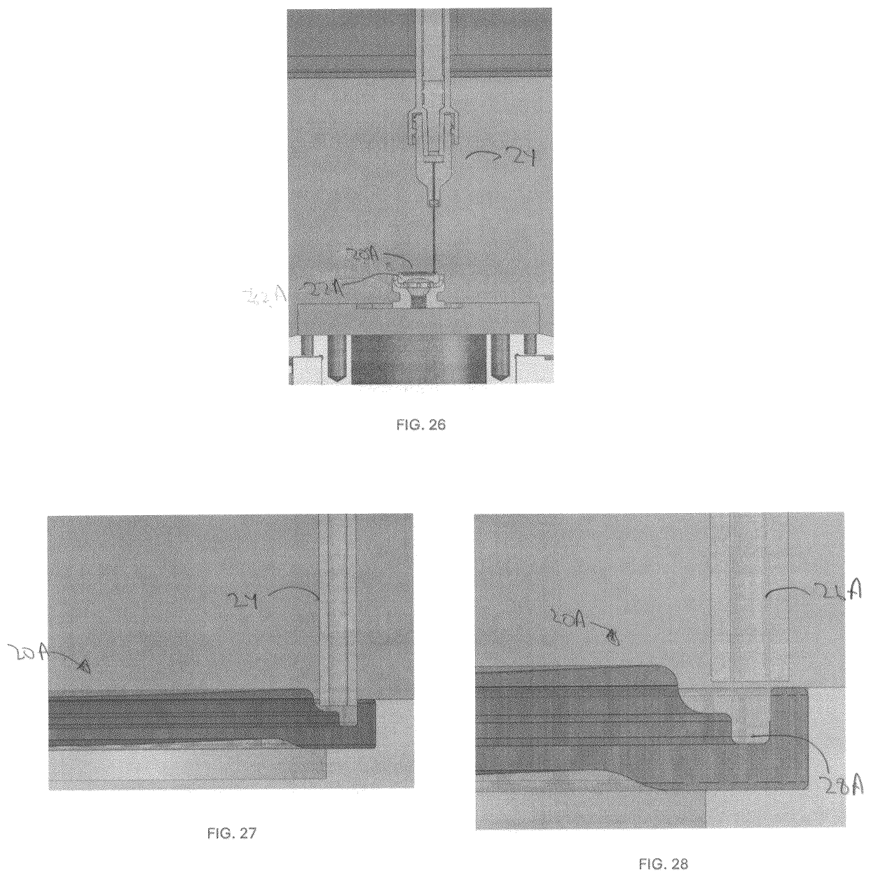

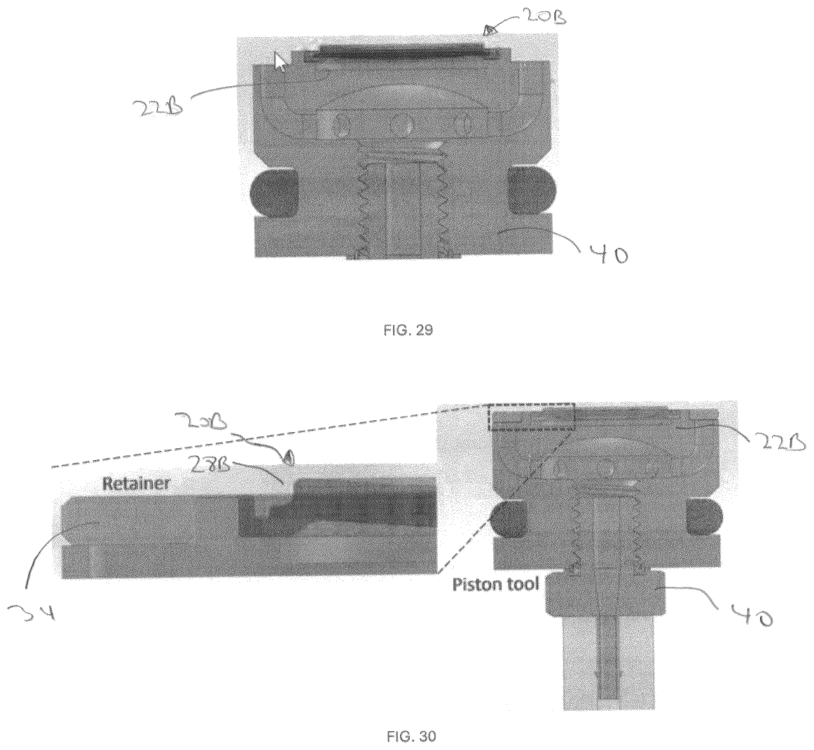

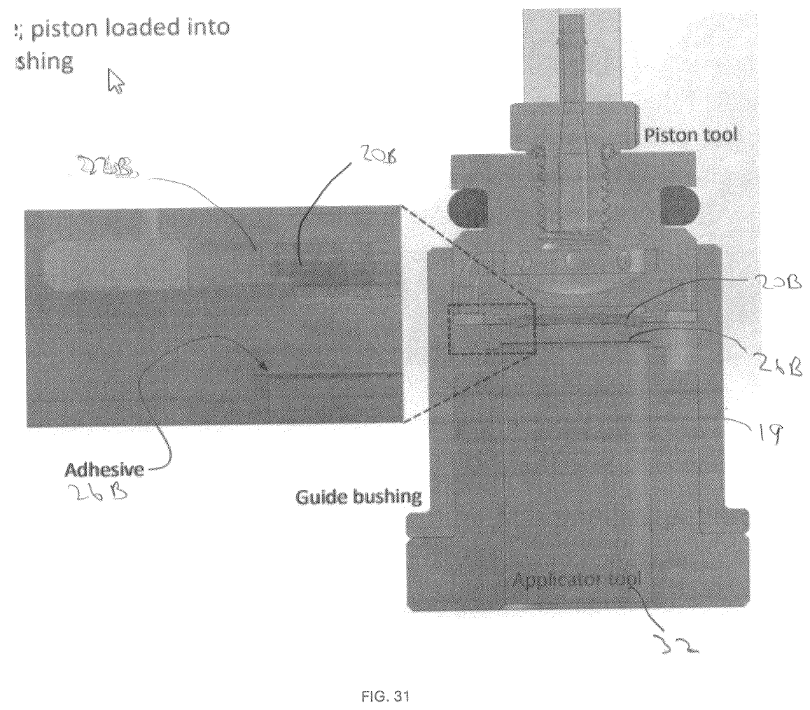

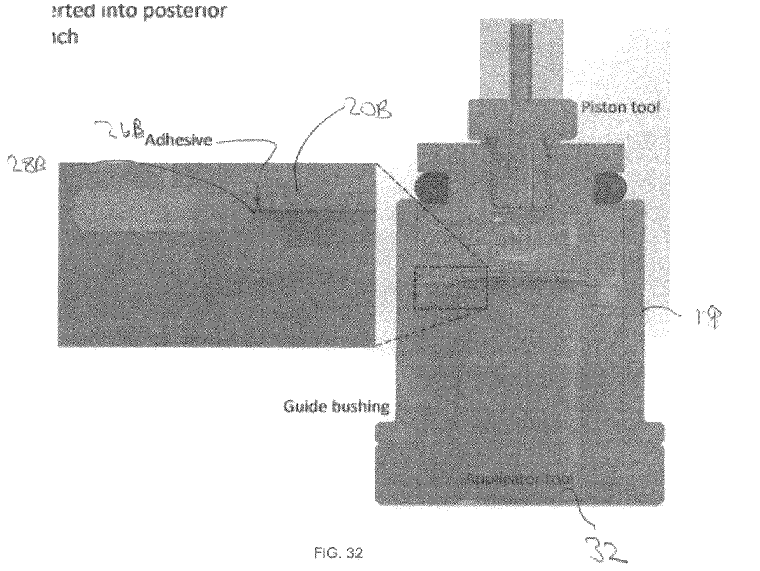

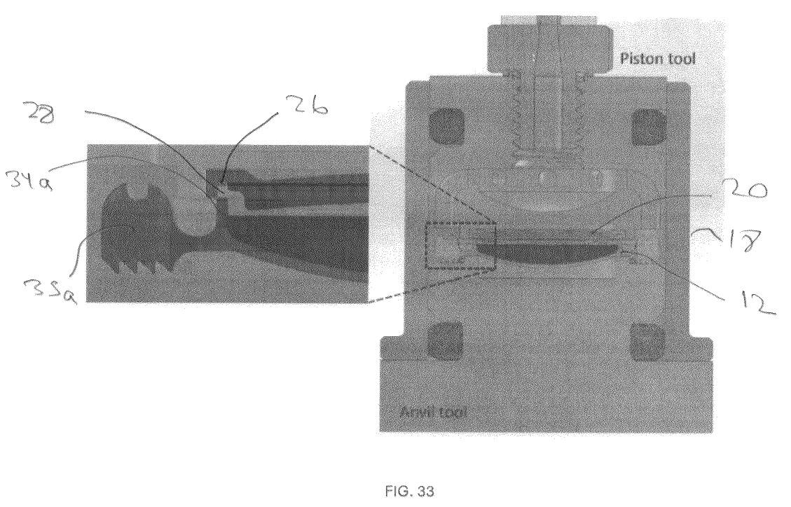

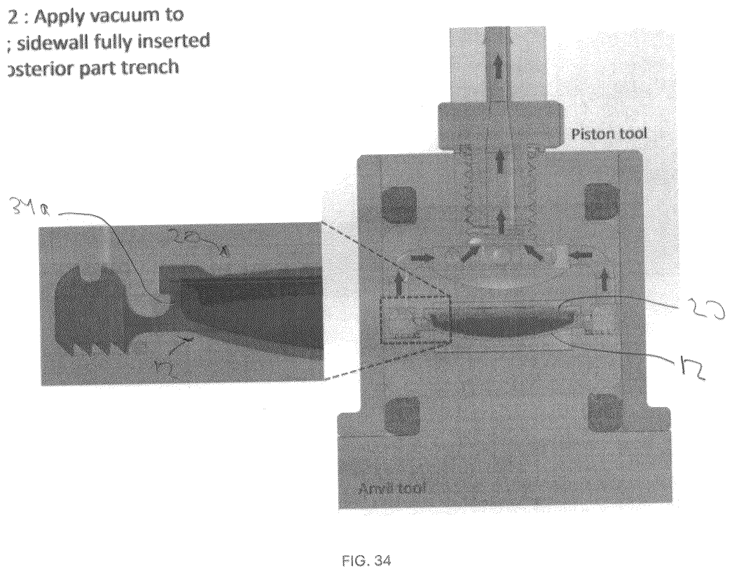

With respect to an example of a method of manufacturing a fluid filled integral IOL, FIG. 22 depicts first nest 10 in an empty state and placed on anvil tool 38. The illustrated nest is depicted as a support structure with protrusions and recesses to support an anterior part of an IOL with a plurality of haptics. However, the nest can comprise other suitable combinations of supporting and fixture features such as openings, cavities, clamps, ribs, grooves, and the like, as appropriate for supporting another configuration of an IOL. FIG. 23 illustrates anterior part 12 of IOL mounted on first nest 10. Anterior part 12 can comprises receptacle 30 defined by anterior face 32 and side wall 34 extending from anterior face and can have at least one haptic 35 extending from the anterior face. As stated above, the anterior part (as well as the posterior part) of the IOL can have the configuration of IOLs described herein or other IOLs. FIG. 24 depicts anterior part 12 after a pre-determined amount of desired fluid 14 has been introduced into receptacle 16 of anterior part 12. Guide bushing 18 can be installed onto first nest 10 before, during or after the pre-determined desired amount of fluid has been introduced into the receptacle of the anterior part of the IOL as illustrated in FIG. 25. A bonding agent can be introduced into the trough of the posterior part of the IOL using a suitable technique. For example, and with reference to FIGS. 26-28, posterior part 20A can be mounted on second nest 22A and injector 24 (which can be, for example, a needle or pump) can be used to inject bonding agent 26A into trough 28A of posterior part 20A. Alternatively, and with reference to FIGS. 29-32, posterior part 20B can be mounted on second nest 22B that is positioned on pistol tool 40 and bonding agent 26B can be deposited on applicator tool 32. Applicator tool 32 can be brought into contact with trough 28B to transfer bonding agent 26B into trough 28B. Referring to FIG. 30, retainer 34 can be positioned within a lateral recess of second nest 22 adjacent to posterior part 20 and sized and configured to precisely align the posterior part with the applicator tool (and can also precisely align the posterior part with the anterior part when such parts are brought adjacent to each other). Further, second nest 22B with posterior part 20B mounted thereon can be inserted into guide bushing 19 as illustrated in FIGS. 31 and 32 to align second nest 22B with applicator tool 32 to ensure the proper and precise transfer of the bonding agent to the trough of posterior part 20B. As illustrated in FIG. 33, second nest 22 with posterior part 20 mounted thereon can be inserted into guide bushing 18 to align posterior part 20 with anterior part 12. As schematically in FIG. 34, suction can be applied to draw second nest 22 towards first nest 10 (or the first nest towards the second nest depending on the direction the suction is applied) such that posterior part 20 and anterior part 12 seal together to form a fluid filled integral IOL. As stated above, other forms of force can be used to bring the second nest and first nest together such as mechanical compression. During this step, the first and second nests can be drawn together and then held in a precisely calibrated position/orientation. This can be accomplished with an internal or external fixture, set screw, or other devices to align and orient the two nests.

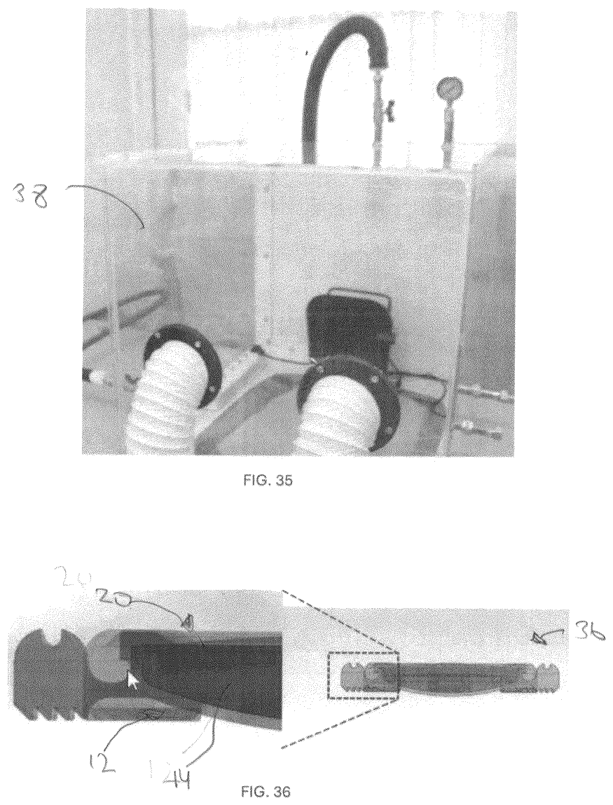

As can be seen in FIG. 34, sidewall 34 of anterior part 12 is fully inserted in trough 28 of posterior part 20. Alternatively, and with respect to FIG. 35, the posterior part and anterior part can be placed into a sealed small inert gas that diffuses readily out of the IOL (e.g. helium) chamber 38. In particular, air can be evacuated from the chamber and the inert gas can be introduced into the chamber and the posterior part and the anterior part can be joined together in the inert gas chamber. The assembled anterior and posterior part can be removed from the chamber and transitioned to another space (such as a heat source like an oven or ultraviolet source). Alternatively heat or UV can be applied while the anterior and posterior parts remain in the chamber during curing. The assembled anterior and posterior parts can be removed (and the bonding agent can be cured for example) and the resulting fluid filled integral IOL 36 is illustrated in FIG. 36.

Such a method of manufacturing has several advantages as described above. By introducing the fluid in the receptacle/chamber of the anterior part of the IOL before sealing the anterior part to the posterior part of the IOL, no modification to the amount of fluid is necessary post-manufacturing. With manufacturing methods where the fluid is introduced in the IOL after the anterior and posterior part are sealed, such fluid may be introduced via a needle or pump that penetrates the side wall of the IOL. In such instances, the needle or pump may retain some of the fluid. Reintroducing fluid may introduce air bubbles into the chamber and evacuating such air may also result in the evacuating needle also retaining some of the fluid. As such, it is difficult to deliver the precise amount of fluid in the receptacle/chamber to obtain the desired power of the lens. The present manufacturing methods allow for a precise delivery of the exact amount of fluid for the exact power of the IOL and removes air from the chamber. Further because the bonding agent, the anterior part, and the posterior part can be fabricated from the same base fabrication material, such as silicone, once the sealant cures, the assembled IOL becomes a single piece integral IOL with the fluid in the receptacle/chamber. Such a manufacturing process allows for precise filling and sealing of the IOL. It also allows for the manufacturing to be scaled by having systems that would allow multiple lenses to be assembled, cured, and sealed simultaneously. The present manufacturing methods can use other components such as nests, bushings, retainers, and anvils, for example, in conjunction with vacuum or an inert gas chamber to precisely align the side wall of the anterior part with the trough of the posterior part such that the orientation of the anterior part and posterior part are correct during assembly.

Each of the disclosed aspects and embodiments of the present disclosure may be considered individually or in combination with other aspects and embodiments as well as with respect to other accommodating intra-ocular lenses, such as IOLs disclosed in U.S. patent application Ser. No. 16/288,723 filed on Feb. 28, 2019 and incorporated by reference in its entirety and U.S. Provisional Application No. 62/842,788 filed on May 3, 2019 and incorporated by reference in its entirety. In addition, orientations of a shape-changing optic can be modified. For example, when implanted, the lens can be flipped such that the anterior face is facing in a posterior direction and the posterior face/optic is facing in an anterior direction. Further, the IOL can be configured such that it is foldable for insertion. Further, while certain features of embodiments may be shown in only certain figures, such features can be incorporated into or deleted from other embodiments shown in other figures or otherwise disclosed in the specification. Additionally, when describing a range, all points within that range are included in this disclosure.

What is claimed is:

1. A method of manufacturing an integral fluid filled IOL comprising an anterior part and a posterior part, the method comprising:
mounting the anterior part of the IOL on a first nest, the anterior part defining a receptacle and having at least one haptic extending from an anterior face thereof;
introducing a pre-determined desired amount of an optical fluid or gel into the receptacle;
introducing a bonding agent into a trough of the posterior part of the IOL;
mounting the posterior part on a second nest;
applying force to draw the first nest towards the second nest or the second nest towards the first nest or placing the first and second nests in a vacuum sealed chamber and mechanically drawing the first and second nest together or placing the first and second nests in an inert gas chamber such that the posterior part and the anterior part are mechanically join together to form a fluid filled integral IOL; and
removing the integral fluid filled IOL from the first and second nests.

2. The method of claim 1, wherein introducing a bonding agent comprises injecting an adhesive, sealant, or other suitable bonding agent into the trough via an injector.

3. The method of claim 1, wherein introducing the bonding agent comprises: depositing the bonding agent on an applicator tool and contacting the bonding agent with the trough to transfer the bonding agent from the applicator tool to the trough.

4. The method of claim 1, further comprising inserting the second nest with the posterior part mounted thereon into a guide bushing to align the second nest with the applicator tool to ensure proper and precise transfer of the bonding agent to the trough of the posterior part.

5. The method of manufacturing according to claim 1, further comprising:
installing a guide bushing onto the first nest before, during or after introducing the pre-determined desired amount of fluid into the receptacle; and
inserting the second nest with the posterior part mounted thereon into the guide bushing to align the posterior part of the IOL with the anterior part of the IOL.

6. The method of claim 1, further comprising a retainer positioned within a lateral recess of the second nest adjacent to the posterior part of the IOL and sized and configured to precisely align the posterior part with the anterior part of the IOL.

7. The method of claim 1, wherein the optical fluid or gel is a silicone oil.

8. The method of claim 1, wherein the anterior part, the posterior part and the bonding agent comprise the same base fabrication material such that the IOL is an integral single-piece IOL after manufacturing.

9. The method of claim 8, wherein the anterior part, the posterior part, and the bonding agent comprise silicone.

10. The method of claim 1, further comprising curing the bonding agent after the posterior part and anterior part have joined together to seal the anterior part and posterior part to form the integral fluid filled IOL.
